(19) United States
(12) Patent Application Publication
(10) Pub. No.: US 2004/0198696 A1
(43) Pub. Date: Oct. 7, 2004

(54) Agent and food for inhibiting IgE antibody

(75) Inventors: Nobukazu Onishi, Hiroshima (JP); Kunihiko Hashimoto, Hiroshima (JP); Hisao Shimizu, Mihara (JP)

Correspondence Address: SUGHRUE MION, PLLC, 2100 PENNSYLVANIA AVENUE, N.W., SUITE 800, WASHINGTON, DC, 20037, US

(73) Assignee: THE JAPAN RESEARCH AND DEVELOPMENT ASSOCIATION FOR N.F.

(21) Appl. No.: 10/826,303

(22) Filed: Apr. 19, 2004

Related U.S. Application Data
(63) Continuation of application No. 10/212,071, filed on Aug. 6, 2002, now abandoned.

(30) Foreign Application Priority Data
Aug. 6, 2001 (JP) P. 2001-237993

(51) Int. Cl. A61K 31/715

(52) U.S. Cl. 514/54

(57) ABSTRACT

Disclosed is an IgE antibody inhibitor containing glucomannan. Also disclosed is an IgE antibody inhibitory food containing glucomannan.

### FIELD OF THE INVENTION

This invention relates to IgE antibody inhibitors and foods, particularly, it relates to an IgE antibody inhibitor and food from which type I allergic disease onset prevention and the like actions can be expected.

### BACKGROUND OF THE INVENTION

Accompanied by the changes in dietary life, residential environment and the like, morbidity rate and mortality rate of allergic diseases are showing a world-wide increasing tendency for the past 10 years. According to a private investigation (“The Present Situation and Future Prospect on New Drug Development” '91 edition, Seed Planning), one in every three people in Japan are currently showing atopic dermatitis, bronchial asthma, allergic rhinitis and the like symptoms of typical type I allergic diseases. These data are also supported by the Investigation on Health and Welfare Trends, the Ministry of Health and Welfare, (1991). Though allergic diseases are rarely concerned in mortal danger directly, they suddenly appear in very younger generations and become chronic because spontaneous cure at early stage can hardly be expected. Accordingly, not only the burden to the patients and their families as a matter of course, but they also exert great influences on social activities for a prolonged period of time.

It is considered that the type I allergic diseases are sensitized and induced by the following mechanism. Firstly, when indoor dust, mite, pollens, fungi and the like antigens are inhaled, B cells release IgE antibodies by the action of CD4 positive T cells which produce Th2 type cytokine. Sensitization is established by further binding to receptors on mast cells at the Fc fragment of IgE antibodies. Next, histamine, leukotriene and the like chemical mediators are released by cross-linking of the Fab fragment of the IgE antibodies on the surface of mast cells by the reinvaded antigens. These substances cause inflammation of tissues, acceleration of vascular permeability, contraction of smooth muscles, acceleration of mucus secretion and the like and thereby induce morbid states of allergic diseases.

The most effective method for treating allergic diseases is to avoid contact with antigens. However, the patients sensitized and induced by antigens which are released and present everywhere in the residential environment have to depend on a temporary resolving means using a symptomatic therapy drug such as an antihistaminic which shows side effects. Onset of the diseases is repeated unless continuing internal use or application of drugs, and there is a fear of worsening the symptoms by rebound when their use is suspended. Because of this, such patients are forced to have great burdens economically and physically.

### SUMMARY OF THE INVENTION

Taking the aforementioned problems into consideration, an object of the invention is to provide an IgE antibody inhibitor and food, which can control IgE antibody titer in vivo, prevent onset of atopic dermatitis, bronchial asthma, allergic rhinitis and the like allergic diseases, and can treat and improve morbid states even when these diseases are induced, and which are safe and easy to intake.

The “living body” addressed herein includes those of warm-blooded animals, preferably mammals, more preferably human.

Other objects and effects of the present invention will become apparent from the following description.

To achieve the above-described objects, the present inventors have conducted extensive studies in order to develop a drug or food having a function to improve morbid states of allergic diseases by inhibiting production of IgE antibodies. As a result, the invention found for the first time that glucomannan has a markedly high IgE antibody inhibitory capacity and a function to prevent allergic diseases. The present invention has been accomplished based on this finding.

That is, the above-described objects of the invention have been achieved by providing the following.

(1) An IgE antibody inhibitor, which contains glucomannan.

(2) The IgE antibody inhibitor described in the above item (1), wherein the glucomannan is in the form of refined konjak flour.

(3) The IgE antibody inhibitor described in the above item (1) or (2), wherein the glucomannan has a dietary fiber content of 95% or more.

(4) The IgE antibody inhibitor described in anyone of the above items (1) to (3), wherein the glucomannan is easily soluble in water.

(5) The IgE antibody inhibitor described in the above item (4), wherein the glucomannan is a pulverized product.

(6) The IgE antibody inhibitor described in the above item (4), wherein the glucomannan has a weight average molecular weight of 1,000,000 or more and an average particle diameter of 100 μm or less, and a period of time until its 1% aqueous solution reaches the viscosity peak at room temperature is within 30 minutes.

(7) The IgE antibody inhibitor described in any one of the above items (1) to (6), having a form of powder, capsule, tablet, pill or granule.

(8) An IgE antibody inhibitory food, which contains glucomannan.

(9) The IgE antibody inhibitory food described in the above item (8), wherein the glucomannan is in the form of refined konjak flour.

(10) The IgE antibody inhibitory food described in the above item (8) or (9), wherein the glucomannan has a dietary fiber content of 95% or more.

(11) The IgE antibody inhibitory food described in any one of the above items (8) to (10), wherein the glucomannan is easily soluble in water.

(12) The IgE antibody inhibitory food described in the above item (11), wherein the glucomannan is a pulverized product.

(13) The IgE antibody inhibitory food described in the above item (11), wherein the glucomannan has a weight average molecular weight of 1,000,000 or more and an average particle diameter of 100 μm or less, and a period of time until its 1% aqueous solution reaches the viscosity peak at room temperature is within 30 minutes.

(14) The IgE antibody inhibitory food described in any one of the above items (8) to (13), having a form of powder, capsule, tablet, pill or granule.

### BRIEF DESCRIPTION OF THE DRAWINGS

FIG. 1 is a graph showing a result of Example 1.

FIG. 2 shows photographs showing skin disease conditions of atopic dermatitis spontaneous onset model mice.

### DETAILED DESCRIPTION OF THE INVENTION

Glucomannan as the main component of the IgE antibody inhibitor and food of the invention has a long period of actually used results as a food material and a food additive particularly in Japan and also has high safety. Accordingly, its continuous internal use is possible.

Though materials of the aforementioned glucomannan are not particularly limited, refined konjak flour and the like refined from konjak tuberous roots and the like are desirable from the viewpoint of easy availability. The refined konjak flour to be used in the invention is described in detail in “Science of Konjak (established in 1993)” edited by Satoshi Okimasu. The terminology “konjak” [kon-nyaku] as used herein means Amorphophallus Konjac, which has hitherto been eaten as food, especially in Japan, and which may be called as “devil's tongue”.

As the aforementioned glucomannan, it is desirable that its dietary fiber content is 95% or more. The method for controlling the dietary fiber content within the above range is not particularly limited, but it is desirable to obtain purified glucomannan by purifying the aforementioned refined konjak flour by an ethanol precipitation method.

Also, it is desirable that the aforementioned glucomannan is easily soluble in water. Though the method for making glucomannan into easily water-soluble property is not particularly limited, a pulverization treatment is desirable from the viewpoint of easy workability.

It is desirable that the glucomannan made into easily water-soluble state by the pulverization treatment as described above has a weight average molecular weight of 1,000,000 or more and an average particle diameter of 100 micrometer or less, and a period of time until its 1% aqueous solution reaches the viscosity peak at room temperature is within 30 minutes.

The IgE antibody inhibitor of the invention may be embodied in any form of powder, gelatin capsule and the like capsules, tablets, pills or granules. Also, it may be used together with a filler or may contain other auxiliary component so long as it does not spoil functions of the IgE antibody inhibitor. Any substance which is harmless to human can be used as the auxiliary component which may be contained.

Intake of the IgE antibody inhibitor is effective generally at an oral dose of from 1 to 50 g/60 kg body weight per day in terms of the effective component thereof (glucomannan).

In the case of allergic diseases caused by excess IgE antibody production, the IgE antibody inhibitor of the invention can be used as an allergic disease protecting agent or allergic disease preventing agent.

In addition, it may be embodied also in a form in which it is contained in general food, namely as an IgE antibody inhibitory food.

In order to obtain an IgE antibody inhibitory food, the purified glucomannan of the invention may be blended in response to the property of respective food, for example in a powdery form with a biscuit-like food. Its minimum concentration in food effective in exerting the effects of the invention is 1% by weight or more in terms of the amount of purified glucomannan.

### EXAMPLES

The present invention will be illustrated in greater detail with reference to the following Examples, but the invention should not be construed as being limited thereto.

### Example 1

Analysis of the Amount of IgE Antibody in sera:

<Test Methods>

As the animal to be tested, a spontaneously induced atopic dermatitis model animal NC/nga mouse (hereinafter referred to as “NC mouse”) [Matsuda H et al.; Int. Immunol., 9, 461 (1997)] was used, and males of 4 weeks of age were purchased from Japan SLC. Using 5 animals of the NC mouse as one group, a basal feed administered group, a test feed 1 administered group, a test feed 2 administered group and a test feed 3 administered group were set, and each group was allowed to feed on the basal feed, test feed 1, test feed 2 and test feed 3 freely for 8 weeks.

Feeding MF (solid feed) manufactured by Oriental Yeast, Co., Ltd. was used as the basal feed. Respective test feed was used by adding 5% by weight of each of the following additives to the basal feed.

The additives to be added to respective feed are shown below.

Test feed 1: refined konjak flour (mfd. by Shimizu Kagaku)

Test feed 2: purified high purity glucomannan having the dietary fiber content of 99% or more (mfd. by Shimizu Chemical, trade name “PROPAL A”)

Test feed 3: finely pulverized purified glucomannan made into easily water-soluble state by applying a pulverization treatment (mfd. by Shimizu Chemical)

Also, data on these additives are shown in Table 1 below.

TABLE 1

| | Refined konjak | Purified | Finely pulverized purified |
|---|---|---|---|
| Measured items | flour | glucomannan | glucomannan |
| Average particle diameter (μm) | 274 | 301 | 99 |
| Viscosity peak reaching time (hr) | 4.0 | 7.0 | 0.5 |
| Viscosity (cpa) | 56,200 | 123,700 | 35,100 |
| Weight average molecular weight | 0.98 × 10⁶ | 1.92 × 10⁶ | 1.90 × 10⁶ |
| Dietary fiber content (%) | 75 | 98.5 | 96.8 |

(Note)
The viscosity peak reaching time and the viscosity were measured by dissolving each sample in an aqueous solution of 25° C. to a concentration of 1%. Also, the viscosity indicates a value after the viscosity peak reaching time.

Blood samples were collected from eye veins of all NC mice at an interval of 2 weeks. The blood samples were centrifuged at 1,700 rpm for 10 minutes to obtain sera. Total IgE antibody titers in the thus obtained sera were analyzed by the sandwich ELISA method.

<Test Results>

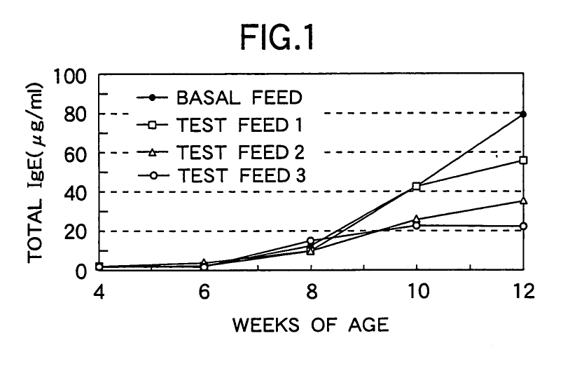

Results of the above test are shown in FIG. 1. The axes of ordinate and abscissa in the drawing respectively show the total IgE content and weeks of age of NC mice.

As shown in FIG. 1, significant increase in the serum IgE antibody content was confirmed until 12 weeks of age in the basal feed administered group. On the other hand, IgE antibody inhibitory action was observed in the groups to which the glucomannan of the invention was administered (test feed 1 administered group, test feed 2 administered group and test feed 3 administered group) when compared with the basal feed administered group. Particularly, the effect was significant in the test feed 2 and 3 administered groups in and after 8 weeks of age. At the time of 12 weeks of age, the IgE antibody production in the test feed 2 administered group was inhibited to about 50% of the basal feed administered group, and about 30% in the test feed 3 administered group.

### Example 2

Observation of Changes in Morbid State in Allergic Disease Model Mice:

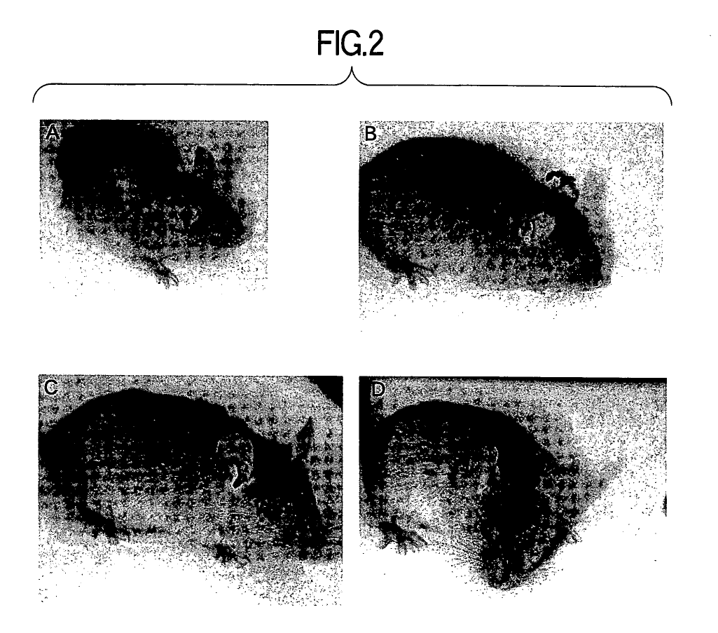

Changes in the morbid state of skin conditions of each of the test animal groups used in Example 1 were observed with the naked eye, with the appearance of the skin disease conditions at the time of 12 weeks of age shown in FIG. 2. The photographs A, B, C and D shown in FIG. 2 are the basal feed administered group, the test feed 1 administered group, the test feed 2 administered group and the test feed 3 administered group, respectively.

In the basal feed administered group and the test feed 1 administered group, loss of hair and bleeding were observed on the cranial region, cervical region and auricle region starting around 9 weeks of age. In addition, deletion of auricle and onset of dermatitis caused by itching behavior were also observed. However, such changes in morbid state were not observed in the test feed 2 administered group and test feed 3 administered group.

As described in the above, the IgE antibody inhibitor and food of the invention can inhibit IgE antibody production in the living body and prevent allergic diseases, because they contain glucomannan. In addition, these effects become more significant by the use of purified glucomannan having a dietary fiber content of 95% or more, more preferably the one which became easily soluble in water by a pulverization treatment, as said glucomannan.

While the invention has been described in detail and with reference to specific examples thereof, it will be apparent to one skilled in the art that various changes and modifications can be made therein without departing from the spirit and scope thereof.

1-14. (canceled).

15. A method for suppressing an allergy comprising administering a pharmacologically effective amount of an IgE antibody inhibitor containing glucomannan to a patient in need thereof,
wherein the glucomannan is in the form of refined konjak flour,
the glucomannan is easily soluble in water, and
the glucomannan is a pulverized product with an average particle diameter of 100 μm or less.

16. A method for suppressing an allergy as described in claim 15, wherein the glucomannan has a dietary fiber content of 95% or more.

17. A method for suppressing an allergy as described in claim 15, wherein the glucomannan has a weight average molecular weight of 1,000,000 or more, and a period of time until its 1% aqueous solution reaches the viscosity peak at room temperature is within 30 minutes.

18. A method for suppressing an allergy as described in claim 15, wherein the IgE antibody inhibitor is in the form of a powder, capsule, tablet, pill or granule.

19. A method for suppressing an allergy comprising ingesting a food containing a pharmacologically effective amount of an IgE antibody inhibitor containing glucomannan,
wherein the glucomannan is in the form of refined konjak flour,
the glucomannan is easily soluble in water, and
the glucomannan is a pulverized product with an average particle diameter of 100 μm or less.

20. A method for suppressing an allergy as described in claim 19, wherein the glucomanan has a dietary fiber content of 95% or more.

21. A method for suppressing an allergy as described in claim 19, wherein the glucomannan has a weight average molecular weight of 1,000,000 or more, and a period of time until its 1% aqueous solution reaches the viscosity peak at room temperature is within 30 minutes.

22. A method for suppressing an allergy as described in claim 22, wherein the IgE antibody inhibitory food on the form of a powder, capsule, tablet, pill or granule.
